(19) United States
(12) Patent Application Publication
(10) Pub. No.: US 2015/0272544 A1
(43) Pub. Date: Oct. 1, 2015

(54) ULTRASONIC PALPATOR, MEASUREMENT SYSTEM AND KIT COMPRISING THE SAME, METHOD FOR DETERMINING A PROPERTY OF AN OBJECT, METHOD FOR OPERATING AND METHOD FOR CALIBRATING A PALPATOR

(71) Applicant: CHARITÉ - UNIVERSITÄTSMEDIZIN BERLIN, Berlin (DE)

(72) Inventors: Kay Raum, Kleinmachnow (DE); Martin Schoene, Berlin (DE); Peter Varga, Davos Platz (CH)

(21) Appl. No.: 14/434,648

(22) PCT Filed: Oct. 9, 2013
(86) PCT No.: PCT/EP2013/071012

§ 371 Date: Apr. 9, 2015

(30) Foreign Application Priority Data
Oct. 9, 2012 (DE) 10 2012 020 075.7

(51) Int. Cl. A61B 8/00; A61B 8/08

(52) U.S. Cl. CPC A61B 8/4281; A61B 8/08; A61B 8/4483; A61B 8/485; A61B 8/58

(57) ABSTRACT

The invention relates to an ultrasonic palpator (1), a measurement system (20) and a kit comprising a palpator (1), a method (40) for determing a propery of an object (4), a method (40) for operating and a method (50) for calibrating a palpator (1). In order to facilitate an easy and precise measurement of the property, the invention provides that a coupler (3) of the palpator (1) is elastically deformable by a pressure (p), which acts between object (4) and coupler (3) during the measurement of the property, and that travel times of ultrasonic sound through the coupler (3) are used for determining the pressure (p).

The invention relates to an ultrasonic palpator with an ultrasonic transducer and an ultrasonic coupler for coupling ultrasonic sound for the ultrasonic transducer into an object of study. Furthermore, the invention relates to a measurement system comprising a palpator and an evaluation apparatus that is connected to the palpator in a measurement signal transmitting manner. Moreover, the invention relates to a kit for examining an object of study with an ultrasonic palpator having an ultrasonic transducer. Further, the invention relates to a method for determing at least one property of an object of study, wherein ultrasonic sound is coupled to the object via a coupling path and travel times of the ultrasonic sound through the object are measured for determining the property. Additionally, the invention relates to a method for operating an ultrasonic palpator for determining at least one property of an object of study, wherein ultrasonic sound is conducted through an ultrasonic coupler and a pressure is excerted onto the object of study by the ultrasonic coupler. Finally, the invention relates to a method for calibrating a palpator, wherein an ultrasonic coupler of the palpator is pressed against a calibration body with a predetermined pressure and ultrasonic sound is conducted through the ultrasonic coupler into the calibration body.

When examining an object of study with ultrasound in order to determine at least one property of the object of study, the property to be determined may change by a pressure with which the ultrasonic coupler is pressed onto the object of study. Determining the pressure requires a separate measurement apparatus, e.g. a strain gage. This separate measurement apparatus, however, increases complexity of the palpator. Furthermore, the additional measurement apparatus cannot exactly determine the pressure acting between the palpator and the object, as it cannot be placed at a contact between the palpator and the object without affecting the measurement results.

Hence, the object of the invention is to provide an ultrasonic palpator, a kit for examining and the methods mentioned above, with which the at least one property of the object of study can be easily and exactly determined.

For the ultrasonic palpator mentioned in the beginning, the object is achieved in that the ultrasonic coupler is elastically deformable. For the measurement system mentioned above, the object is achieved in that the palpator is a palpator according to the invention. The object is achieved for the kit mentioned in the beginning in that the kit comprises at least two ultrasonic couplers for coupling ultrasonic sound from the ultrasonic transducer into the object, wherein the ultrasonic couplers are elastically deformable, have different elasticities and are exchangeably mountable to the palpator. For the method for determining the at least one property, the object is achieved in that a force acting on the object is measured by determining travel times of the ultrasonic sound through the coupling path whose length is changed by the force. For the method for operating the palpator mentioned above, the object is achieved in that the palpator is a palpator according to the invention and in that the ultrasonic coupler is elastically deformed by the pressure and travel times of the ultrasonic sound required for travelling through the ultrasonic coupler are used for determining the pressure. Finally, the object is achieved for the method for calibrating the palpator mentioned above, in that the pressure is varied, wherein the ultrasonic coupler is elastically deformed by the pressure and travel times of the ultrasonic sound required for travelling through the ultrasonic coupler are correlated with the pressure.

By using the elastic ultrasonic coupler, a pressure that is excerted via the coupler onto the object deforms the coupler along a direction in which the pressure acts. By deforming the coupler, a dimension and e.g. a length of the couple and therefore the coupling path that extends through the ultrasonic coupler is shortened. Hence, compared to travel times of ultrasonic sound through a coupler that is not deformed, the travel times of the ultrasonic sound through the deformed coupler are shorter. By using the difference between the travel times, the pressure acting onto the object and therefore acting onto the coupler, too, can be estimated without the need of an additional measurement apparatus. The elastic coupler is deformed by the pressure and has its original form after the pressure is gone.

The solutions according to the invention can be combined as desired and further improved by the further following embodiments, that are advantages on their own in each case.

According to a first possible embodiment, the ultrasonic coupler is elastically deformable by forces acting during the determination of the at least one property of the object of study. In particular, the elasticity of the coupler may essentially resemble the elasticity of the object of study. For instance, the ultrasonic palpator may be an examination probe for determining at least one mechanical property, e.g. the thickness of an elastic material, for instance cartilage of a joint as the object of study. The ultrasonic coupler, which may be connected to the ultrasonic transducer in an ultrasonic sound coupling manner, may be pressed against the object of study during determining of the at least one property, in order to ensure a proper contact between the palpator and the object, such that ultrasonic sounds can be coupled into the object via the coupler effectively. The transducer may emit ultrasonic sound with a maximum at a sound frequency of 20 MHz.

In order to be able to easily deduce deformation of the object due to the force, the elasticity or stiffness of the coupler and of the object may be comparable. For instance, the ultrasonic coupler may have a Young's module between 1 and 10 N/mm² and for instance of 6.5 N/mm² at temperatures between 20° C. and 36° C. The coupler may at least sectionwise or in particular completely have said Young's module. Hence, the ultrasonic coupler preferably has an elasticity that essentially corresponds to the elasticity of the object, e.g. of cartilage, for instance hyaline cartilage, in case cartilage thickness shall be determined. For instance, the at least one property of cartilage may be of interest in case the joint is to be examined for osteoarthritis. A sample of the joint comprising cartilage as the object of study and bone material supporting the cartilage may be taken by surgery before the methods for determining the at least one property or for operating the palpator are applied to the object. In the alternative, the at least one property of cartilage damaged due to osteoarthritis and/or the at least one property of healthy cartilage that shall be used to replace the damaged cartilage can be determined in vivo, hence intraoperative and without taking samples from the joint. The at least one property may be determined by direct examination of the cartilage, i.e. by directly contacting the cartilage. Alternatively, the at least one property can be determined by indirect examination of the cartilage, i.e. by contacting the skin covering the cartilage.

Preferably, the ultrasonic coupler is at least sectionwise made of an elastomer. At least the palpation end of the coupler may be made of an elastomer. Moreover, in order to avoid interfaces between different materials, which may cause unintentional reflections of ultrasonic sound, the ultrasonic coupler may be homogeneously made of an elastic material, for instance the elastomer. The elasticity of elastomer can easily be adapted when forming the coupler. Elastomers are almost incompressible (Poisson's ratio of about 0.5), so an axial compression results in a lateral expansion without a change in volume. Furthermore, elastomers can be deformed essentially without compression. Hence, undeformed and deformed elastomers essentially comprise the same speed of sound, which further facilitates an easy measurement.

It may be necessary to adjust the coupler and e.g. the elastomer in its mechanical and acoustical properties to the object, e.g. cartilage. This requires a fine tuned stiffness, such that the strain of the coupler and the object layer are similar. Furthermore, the effects of geometry of the coupler on the apparent elasticity or stiffness and sound propagation of the coupler may have to be considered. The elasticity or stiffness can be varied by using different types of elastomer, for instance Elastosil made by Wacker Chemie AG, with different Shore-A stiffness values offered by the producer, or by adding aluminum oxide powder during the molding process of the coupler. The mechanical properties of the coupler may be investigated with unconfined compression tests and uniaxial tension tests. These tests clarify the elasticity of the elastomers, especially check for non-linear and viscoelastic behavior.

The cartilage layer compression with the coupler can be assumed as an elastic two-layer system. If the mechanical behavior of the first layer—the coupler—is known, the behavior of the second layer—the object—can be precisely extracted.

Additionally, temperature-dependent and time-dependent (viscoelastic) behavior of the coupler may be investigated. The mechanical behavior of the coupler may be temperature dependent, in case the elasticity of the coupler results from an elastomer. To avoid errors, the coupler should be investigated in the range of room temperature to body temperature (20°-36° C.).

The biomechanics of cartilage are very time dependent, e.g. caused by the biphasic behavior of hyaline articular cartilage. The faster the cartilage is compressed, the higher is the apparent stiffness. After compression, the cartilage relaxes, which is a time-dependent process, too. In order to investigate this behavior with the palpator, it may be useful to quantify the presumable time-dependent behavior of the coupler. For full understanding, the acoustic properties of the coupler may be investigated too. This includes speed of sound, acoustic impedance and attenuation.

The coupler preferably has a sound attenuation and a sound velocity similar to the object, e.g. cartilage. To be able to easily measure not only the length of the measurement path, i.e. the coupler, but also the thickness of the object, the coupling path is preferably long enough such that ultrasonic sound reflections from an interface of the object that faces away from the coupling path can be detected before the first multiple reflection from an end of the coupling path that faces the object can be detected. On the other hand, the length of the coupling path shall be short enough to prevent too strong sound attenuation.

The ultrasonic coupler may be formed with an ultrasonic emitting section that comprises a free emitting or palpation end, wherein the emitting section has a cylindrical shape. The free emitting or palpation end is put directly or with another ultrasonic sound conductive material therebetween on the object of study during operation of the palpator. Due to the cylindrical shape, a good contact between the coupler and the object can be achieved. Furthermore, in case the ultrasonic emitting section is formed as a right cylinder, e.g. a right circular cylinder, it can be easily deformed along its longitudinal axis by forces acting between the coupler and the object during the measurement. The chosen cylindrical shape can cause an almost linear relationship between the force and the amount of elastical deformation of the coupler.

The coupler can be formed with an ultrasonic receiving section that is arranged between the emitting section and the ultrasonic transducer. The receiving section receives ultrasonic sound emitted by the transducer and conducts the ultrasonic sound to the emitting section. Preferably, the receiving section is less deformable than the emitting section and for instance essentially not deformed by forces acting during the measurement in order to facilitate a stable connection between the conductor and the transducer. In order to prevent deformation of the receiving section, the geometry of the receiving section may differ from the geometry of the emitting section. For instance, the receiving section may have a conical shape, e.g. with a trapezoidal cross-section along the longitudinal direction, wherein a wider base surface of the receiving section faces away from the emitting section and a smaller base surface of the receiving section is in contact with the emitting section. Perpendicular to the longitudinal direction, the coupler and in particular its receiving section and/or its emitting section may have a circular cross section, which provides for a homogeneous transfer of the pressure. Again, the ultrasonic coupler may be homogeneously made of one material, wherein the emitting and the receiving sections have different cross-sectional shapes.

In order to further stabilize the form of the receiving section, the receiving section may rest against a housing of the ultrasonic palpator, e.g. perpendicular to the longitudinal direction of the coupler. In particular, if the coupler is made of an elastomer, the receiving section that is supported by the housing cannot readily be deformed by the forces acting during the measurement. Elastomers, namely, are almost incompressible, such that a deformation of the receiving section along the force and in particular in a direction perpendicular to one of its base surfaces would result in an evasive movement of the elastomer perpendicular to the force. The evasive movement, however, may be reduced by the chosen shape of the receiving section and may even be prevented by the housing, against which the receiving section preferably rests.

In order to be able to examine different objects, which may have different elasticities or stiffnesses, the ultrasonic coupler may be repeatedly mounted to the probe. Thus, ultrasonic couplers, whose mechanical properties, i.e. whose stiffnesses or elasticties are adapted to those of the object, can be used, e.g. with the kit according to the invention. For instance, couplers with different mechanical properties, e.g. stiffness or elastic modulus, can be used in different pressure ranges.

For measuring travel times, the palpator preferably comprises an ultrasonic sensor that is connected to the ultrasonic coupler in an ultrasonic coupling manner. The ultrasonic sensor may be a sensor that is formed separate from the ultrasonic transducer. Preferably, however, the ultrasonic transducer can be operated in an ultrasonic sound emitting mode or in a sensor mode, in which the transducer receives ultrasonic sound and generates a measurement signal.

As the travel times depend on the length and the speed of sound of the coupler, the travel time can be calculated by dividing twice the length of the coupler by its speed of sound. Sound reflections occur at interfaces between materials with different acoustic properties, e.g. impedances. Provided the surfaces of the two materials at which they form the interface are smooth, plane and parallel to each other, different frequencies of the ultrasonic sound are equally reflected. If the ultrasonic sound comprises a direction of propagation that is not perpendicular but at an angle to the interface, interferences may occur that amplify attenuate ultrasonic sound at different frequencies.

In order to be able to ascertain a proper connection between the palpator and the object, the palpator may comprise a spectrum analyzer for analyzing the frequency spectrum of ultrasonic sound received by the ultrasonic sensor. In particular if the coupler is tilted with respect to a good measurement position, frequency dependent constructive and/or destructive interference of reflections of the ultrasonic sound occurs and can be detected by the spectrum analyzer.

Based on the detected spectrum, the quality of the contact between the palpator and the object can be determined immediately and the position of the conductor can be corrected to improve measurement quality.

In case the ultrasonic palpator is part of the measurement system, the evaluation apparatus may be adapted to determine a pressure that acts onto the ultrasonic coupler by measuring travel times of ultrasonic sound through the ultrasonic conductor. Furthermore, the spectrum analyzer may be part of the evaluation apparatus instead of the palpator. Hence, such a palpator may be designed very small and ergonomic due to the low amount of components.

When performing the method, frequencies of ultrasonic sound may hence be received, e.g. from the ultrasonic coupler, and be used for determining the quality of a contact of the palpator to the object of study.

The deformation of the object may be correlated with the pressure in a time-resolved manner, e.g. in case the ultrasonic sound is generated in pulses with a sufficient high repetition rate. Furthermore, using and precisely measuring different pressures as well as deformations of the object allows for determining not only the thickness of the object, but also other mechanical properties, such as its stiffness or elastic modulus. For this purpose, the gradient or slope of the change or deformation in dependence of the pressure can be determined based on the measured thicknesses and pressures.

The thickness of the object can be calculated by multiplying the travel time of the speed of sound there and back through the object with the speed of sound and the factor 0.5. The deformation or strain of the object can be calculated by subtracting the travel time difference of the travel time through the object in an undeformed state from the travel time difference of the object, on which the pressure acts and by dividing the result by said travel time difference through the object in its undeformed state.

Often, it is the pressure acting onto the object and the resulting strain in the object that are of interest. The pressure can be calculated by the ratio of the force applied to the object, e.g. via the coupler, and the size of a contact are between the coupler and the object. The contact area may be defined by the size of the emitting end that rests on the object during the determination of the at least one property. Thus, the pressure equals the force divided by the size of the contact area. The strain equals the relative change of thickness of the object in response to the pressure. Therefore, the strain can be calculated by calculating the difference between the thickness of the unstressed object and the object on which the pressure is exerted.

Furthermore, the difference has to be divided by the thickness of the unstressed object in order to calculate the strain.

For solid bodies, the Hooke's law may be applied, according to which the stress or force can be calculated by multiplying the strain with the Young's modulus of the deformed material, e.g. the coupler of the object. However, not all materials follow Hooke's law. For instance rubber-like materials do not comprise a linear pressure-strain behavior, but follow a hysteresis curve when pressure increasingly acts onto the rubber-like material and is subsequently reduces.

For experiments on native cartilage usually the thickness is not known and needs to be estimated by suitable techniques. Common techniques are optical techniques, needle probe, magnetic resonance imaging (MRI) or ultrasound. Optical methods use either histological sections, which means a destruction of the sample, or optical coherence tomography (OCT) which is non-destructive. Current MRI techniques are very time-consuming and expensive and achieve not the same accuracy like other methods due to the limited voxel size. The needle technique uses a needle which is pressed into the cartilage and displacement and force are recorded simultaneously. At that point the needle penetrates the cartilage surface a first peak in the force can be detected, a second peak when the needle hits the beginning of the mineralized tissue. The spatial distance between the peaks give the cartilage thickness. Disadvantages of this method are the local penetration and the lack of standardizations. The ultrasound method uses the temporal delay between the pulse echoes of the cartilage surface and the cartilage-bone interface. To be able to get the thickness the speed of sound of the cartilage has to be known. In the most studies this value is merely assumed, which increases the risk of errors. Nevertheless this method enables cartilage thickness estimation non-destructively with sufficient accuracy.

The biomechanics are usually described with the biphasic model, sometimes with the tri-phasic model. According to the biphasic model articular cartilage consist of a solid phase (proteoglycans, type 2 collagen, chondrocytes) and a fluid phase (water). The interstitial water can move through the solid matrix of the cartilage layer, but that has a low permeability, so there is a high resistance against the flow. This results in a time-dependent mechanical behavior. Directly after a cartilage compression, which takes around 1 second, the stress at the deformation area increases, which results in a pressure gradient. The compensation of the pressure gradient (relaxation) is delayed by the low permeability and takes around 10 minutes. Several studies tried to characterize the biomechanics of cartilage only by the stress-strain curves at the equilibrium state. This allows only characterization of the solid phase and is hardly comparable to normal physiological loading frequencies, like occurring while walking.

The invention will be described hereinafter in greater detail and in an exemplary manner using advantageous embodiments and with reference to the drawings. The described embodiments are only possible configurations in which, however, the individual features as described above can be provided independently of one another and can be omitted in the drawings:

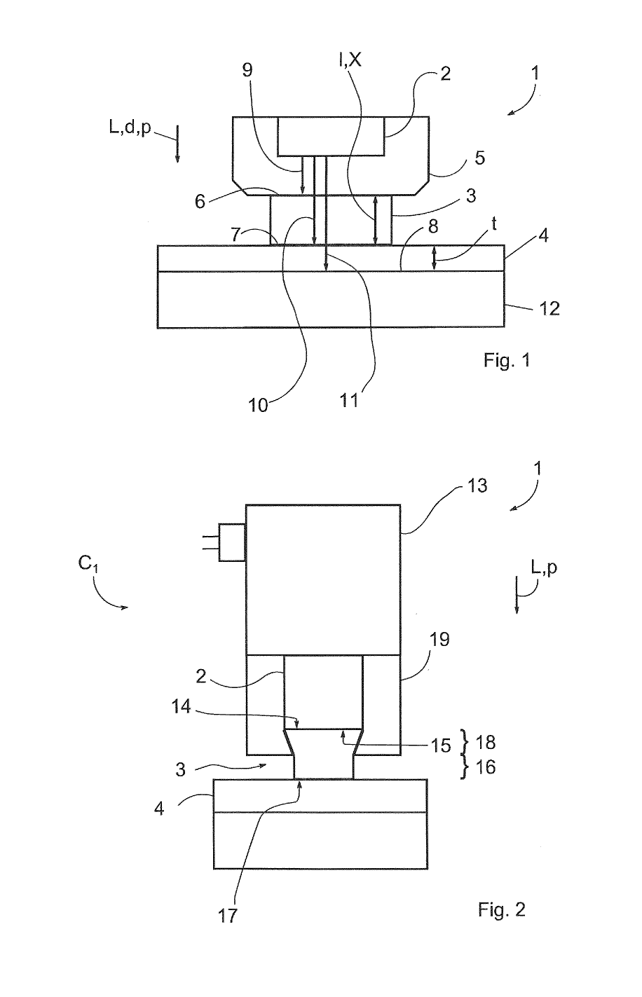

FIG. 1 is a schematic cross-sectional view of an exemplary embodiment of the ultrasonic palpator;

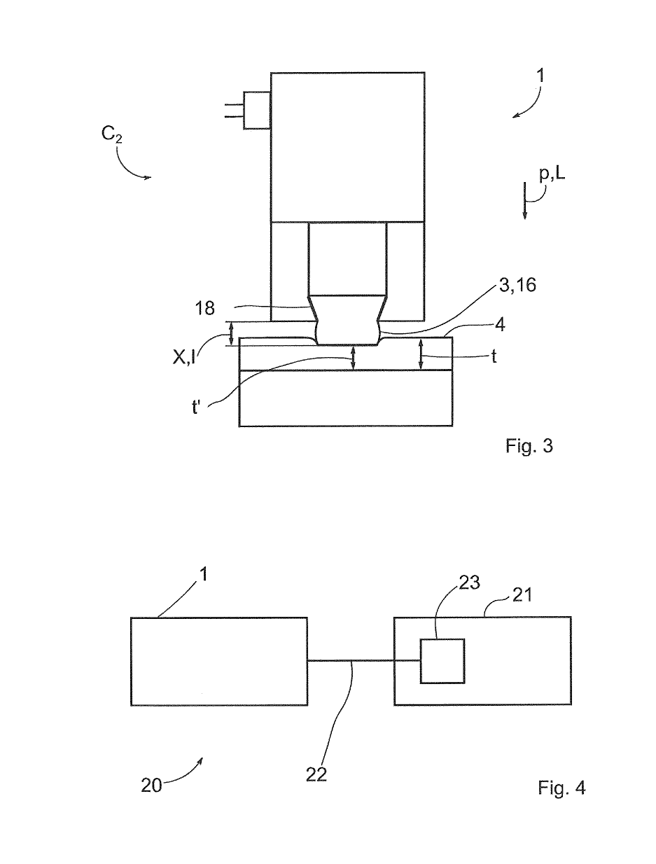

FIGS. 2 and 3 are schematic cross-sectional views of another exemplary embodiment of the ultrasonic palpator;

FIG. 4 shows a schematic view of an exemplary embodiment of a measurement system;

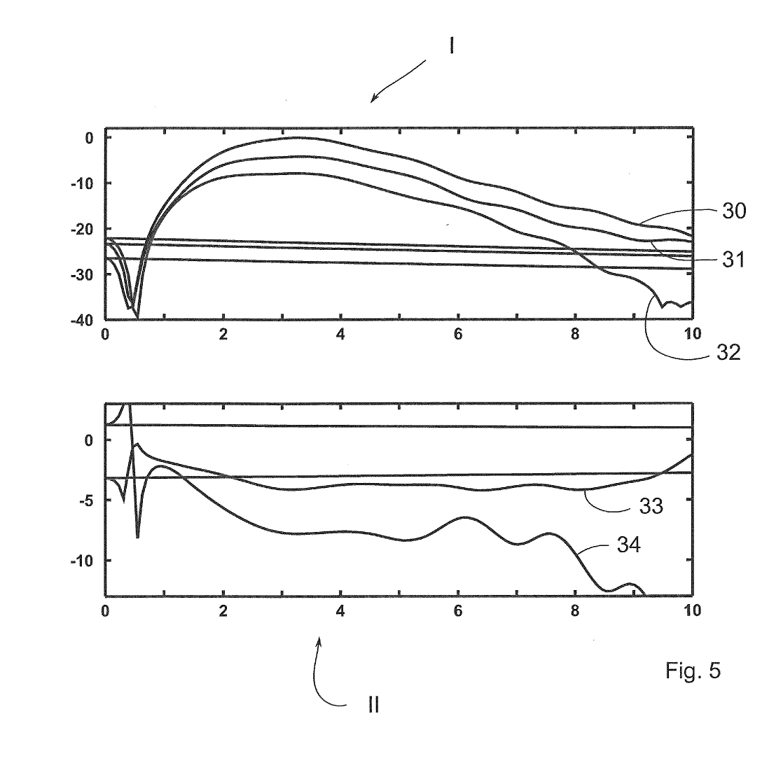

FIG. 5 schematically shows frequency spectra of ultrasonic sound;

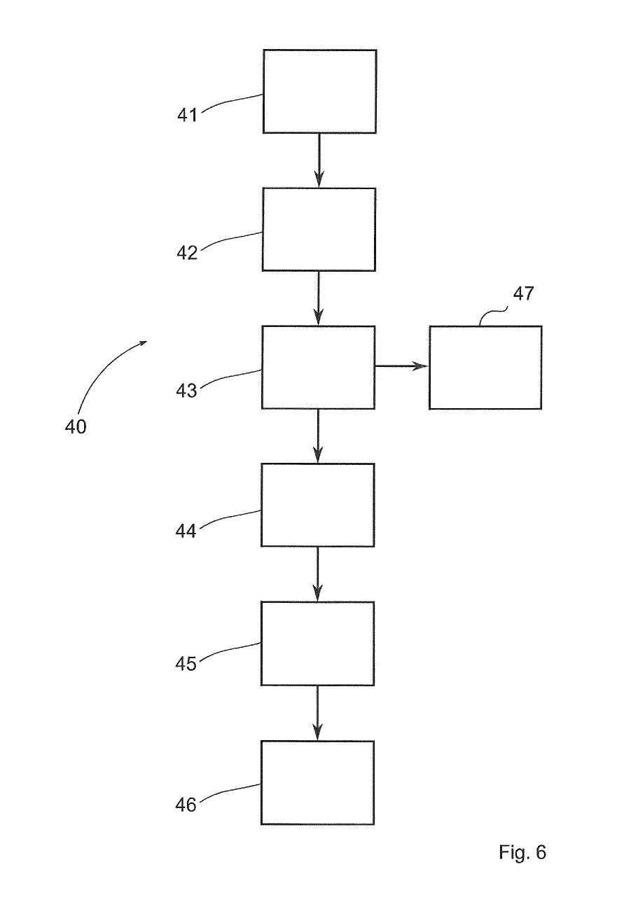

FIG. 6 shows an exemplary embodiment of a method for determining at least one property of an object of study, e.g. by operating one of the palpators according to the previous exemplary embodiments; and

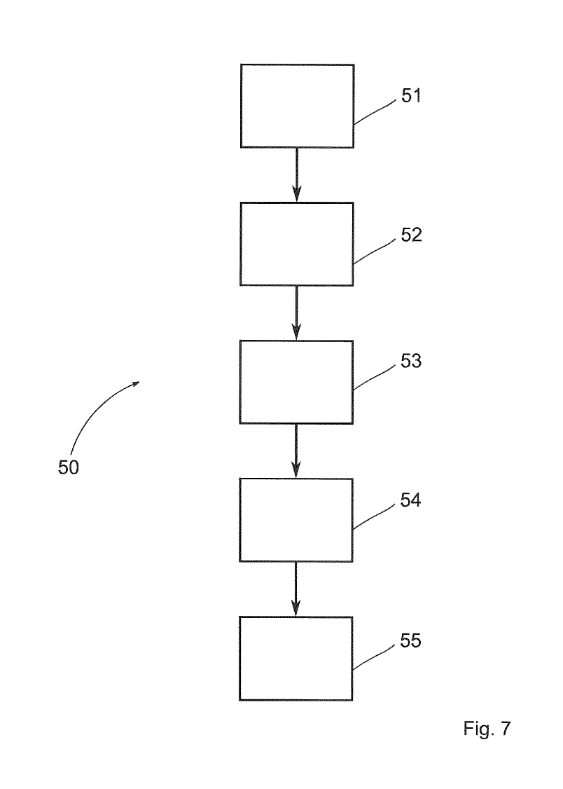

FIG. 7 schematically shows an exemplary embodiment of a method for calibrating a palpator.

First, an ultrasonic palpator 1 is described with reference to FIG. 1. The palpator 1 comprises an ultrasonic transducer 2, which emits ultrasonic sound when the palpator 1 is operated. Furthermore, the palpator 1 comprises an ultrasonic coupler 3 for coupling ultrasonic sound, emitted from the transducer 2, into an object of study 4. Between the transducer 2 and the coupler 3, an ultrasonic conductor 5 is arranged, which conducts ultrasonic sound from the transducer 2 to the coupler 3 and vice versa. The ultrasonic sound is depicted as arrows 9, 10, 11, which end at interfaces 6, 7, 8 between the conductor 5 and the coupler 3, the coupler 3 and the object 4 and the object 4 and a base material 12, on which the object 4 is arranged.

In order to ascertain a good ultrasonic conductivity between the palpator 1 and the object 4, the coupler 3 is pressed against the object 4 with a pressure p. At least at the interface 7 between the coupler 3 and the object 4, the pressure p is parallel to a direction of propagation d of the ultra sound 9, 10, 11. Along the direction d, the transducer 2, the conductor 5 and the coupler 3 may be arranged after each other.

The coupler 3 is elastically deformable by the pressure p and is in particular deformable along its longitudinal direction L. If the coupler 3 is pressed against the object 4 a length I of the coupler 3 and hence a coupling path, along which the ultrasonic sound 10 travels at least through the coupler 3, is reduced, such that a distance between the transducer 2 and the interface 7 is reduced due to the deformation. Hence, ultrasonic sound reaches the interface 7 earlier compared to the case, in which no forces act onto the coupler 3. Due to the time difference, the pressure p can be determined. For instance, the length I of the undeformed coupler 3 may be between 2,5 mm and 25 mm, in particular between 5 mm and 20 mm and for instance 5 mm or 11 mm.

The object of study 4 may be elastic and for instance cartilage, in particular hyaline articular cartilage of a joint. The pressure p, thus, not only deforms the coupler 3 but also the object 4. The base material 12 supporting the object, e.g. bone material supporting the cartilage, is essentially undeformable by the pressure p and may be a bone underling the cartilage. When determining the at least one property, in particular a mechanical property and for instance a thickness t of the object 4, the pressure p deforms the object 4, such that the thickness may be reduced. Hence, without knowledge of the pressure and without knowledge about the mechanical properties, e.g. of the stiffness or flexibility of the object 4, the thickness t of the object 4 can be determined with the palpator by knowing the speed of sound. The mechanical properties like stiffness or elasticity can be determined or known before the thickness t is measured. The pressure p can be determined during the measurement of the thickness t.

FIG. 2 shows another exemplary embodiment of the ultrasonic palpator 1 in a schematic cross-sectional view. Same reference signs are being used for elements, which correspond in function and/or structure to the elements of the exemplary embodiment of FIG. 1. For the sake of brevity, only the differences from the exemplary embodiment of FIG. 1 are looked at.

The ultrasonic palpator 1 is shown with a housing 13, which may comprise further electronics, e.g. for driving the transducer 2 or for determining travel times of ultrasonic sound through the coupler 3 and/or the object 4.

Transducer 2 directly contacts coupler 3, in order to reduce the number of interfaces and to more effectively conduct sound from the transducer 2 to the coupler 3. The coupler 3 may for instance be glued to the transducer 2, for instance by PMMA (polymethylmethacrylat) or other suitable materials. Alternatively, a receiving end 14 of the coupler 3 may rest against a transmitting end 15 of the transducer 2 and may be pressed against the transmitting end 15 without a material fit interconnecting transducer 2 and coupler 3.

The coupler 3 is formed with an ultrasonic emitting section 16, which has a cylindrical shape and for instance a right circular cylindrical shape. The ultrasonic emitting section 16 protrudes e.g. 2 mm from the housing 13 and is readily accessible perpendicular to its longitudinal direction L. A free end 17 of the emitting section 16, which may be designated as a palpation end, may be plane and be pressed against the object 4 during a measurement. The ultrasonic emitting section 16 protruding from the housing 13 may be designated as unconfined section. A length or thickness of the undeformed ultrasonic emitting section 16 parallel to the length I may be between 1 mm and 10 mm and in particular between 2 mm and 8 mm, for example 2 mm or 5 mm.

Between the emitting section 16 and the transducer 2, the coupler 3 comprises an ultrasonic receiving section 18, which is less deformable than the emitting section 16. For instance, the receiving section 16 may have a different shape and may for instance be conical or have a trapezoidal cross-section. A wider base of the receiving section 18 forms the receiving end 14 of the coupler 3. The coupler 3 comprising the emitting section 16 and the receiving section 18 may be free from internal interfaces, such that ultrasonic sound can be led through the coupler 3 without reflections.

Additionally or alternatively to the shape for reducing elasticity of the receiving section 18, the receiving section 18 may be braced or supported perpendicular to the longitudinal direction L. In case the coupler 3 is made of an elastomer, bracing the receiving section 18 perpendicular to its longitudinal direction L reduces deformability of the receiving section 18. Elastomers, namely, cannot be compressed, but only be deformed. Hence, when the pressure p acts parallel to the longitudinal direction L and seeks to deform the receiving section 18, the receiving section 18 at least partially would react with an evasive movement, which, however, is blocked or prevented by the housing 14. The housing 14 may comprise a holding section 19, in which the transducer 2 and the coupler 3 are at least sectionwise arranged. In particular, the receiving section 18 may be arranged in the holding section 19. The receiving section 18 that is braced or supported and e.g. arranged in the housing 14 may designated confined section. A length or thickness of the undeformed receiving section 18 parallel to the length I may be between 1,5 mm and 15 mm and in particular between 3 mm and 12 mm, for example 3 mm or 7,5 mm.

FIG. 2 shows the ultrasonic palpator 1 in a condition C1, in which no pressure p acts between the coupler 3 and the object 4.

FIG. 3 shows the palpator 1 of the exemplary embodiment of FIG. 2 in a condition C2, in which the coupler 3 is pressed onto the object 4 with a pressure p and along its longitudinal direction L. Compared to the condition C1 of FIG. 4, the length I of the coupler 3 and in particular of its emitting section 16 and thus the coupling path X is reduced due to the pressure p. Due to the reduction of length I the emitting section 16 curves or bulges perpendicular to the longitudinal direction L, such that the emitting section 16 has a barrel-shape. The receiving section 18, on the contrary, is not deformed.

Furthermore, due to the pressure p the thickness of the object 4 is reduced to thickness t′, which is less than the thickness t of the object of areas on which no pressure p acts.

FIG. 4 shows an exemplary embodiment of a measurement system 20 in a schematic view. Same reference signs are being used for elements, which correspond in function and/or structure to the elements of the previous exemplary embodiments. For the sake of brevity, only the differences from the exemplary embodiments of FIGS. 1 to 3 are discussed.

The measurement system 20 comprises the palpator 1 and an evaluation apparatus 21. The evaluation apparatus 21 is connected to palpator 1 in a measurement signal transmitting manner, for instance by a signal line 22. The evaluation apparatus 21 and the palpator 1 may be formed integrally. Alternatively, the evaluation apparatus 21 may be a separate apparatus, for instance a computer, e.g. a laptop computer or a dedicated computer.

The evaluation apparatus 21 is adapted to measure travel times of ultrasonic sound, in particular through the coupler 3 and the object 4. Based on the travel times of ultrasonic sound through coupler 3, the evaluation apparatus 21 can determine the pressure p, which acts between the coupler 3 and the object 4. Furthermore, the measurement system 20 may comprise a spectrum analyzer 23 for analyzing the spectrum of ultrasonic sound represented by the measurement signal transmitted by signal line 22 that can be part of the evaluation apparatus 21. The spectrum analyzer 23, however, may be part of the palpator 1 instead of being part of the evaluation apparatus 21.

FIG. 5 shows spectra of ultrasonic sound reflected by interface 7 between the coupler 3 and the object 4. Same reference signs are being used for elements, which correspond in function and/or structure to the elements of the previous exemplary embodiments. For the sake of brevity, only the differences from the exemplary embodiments of FIGS. 1 to 4 are discussed.

The upper graph I shows three spectra 30, 31, 32 of ultrasonic sound reflected by interface 7. Lower graph II shows difference spectra 33, 34. Spectrum 30 represents a reference spectrum, in which the contact at interface 7 is perfect. Spectrum 30 may be determined but not placing the coupler 3 against the object 4 or any other object, such that the coupler 3 merely contacts air. Spectrum 31 represents a spectrum for a good contact at interface 7. Spectrum 32 is a spectrum with an insufficient contact at interface 7. For instance, coupler 3 may be tilted when measuring spectrum 32 and with respect to a good measurement position, in which spectrum 31 may be determined. Difference spectrum 33 is the difference between spectra 30 and 31. Difference spectrum 34 is the difference spectrum of spectra 30 and 32.

Based on the spectra 30, 31, 32 and/or their difference spectra 33, 34, the quality of the contact at interface 7 between coupler 3 and object 4 can be determined. In case of an insufficient contact, the measurement cannot reliably be performed. Hence, when analyzing the spectra, the measurement quality of the thickness t of object 4 can be further improved. A quality signal representing the quality of contact between coupler 3 and object 4 can be generated in dependence of the spectra 31, 32 and/or the difference spectra 33, 34.

FIG. 6 shows an exemplary embodiment of a method for determining at least one property of the object 4, e.g. by operating the palpator 1 as a flow chart. Same reference signs are being used for elements, which correspond in function and/or structure to the elements of the previous exemplary embodiments. For the sake of brevity, only the differences from the exemplary embodiments of FIGS. 1 to 5 are discussed.

Method 40 starts with a first method step 41. For instance, palpator 1 may be put into operation in method step 41. In method step 42, which follows step 41, coupler 3 is pressed against object 4 with an unknown pressure p. After step 42, step 43 follows, in which ultrasonic sound is coupled into the object 4 via coupler 3, which forms the coupling path X that extends through the coupler 3 along its longitudinal direction L. For instance, the ultrasonic sound may be emitted as ultrasonic pulses. The repetition rate of the pulses may be high enough to be able to measure time dependencies of the deformation of the object in response to the precisely measured pressure.

In the next method step 44 travel times of ultrasonic sound through the coupling path X, i.e. through the coupler 3 or between the transducer 2 and the interface 7, are measured. Based on the travel times measured in step 44, the pressure p is determined and e.g. calculated in the evaluation apparatus 21 in method step 45. In method step 46 method 40 ends. For instance, the thickness t′ and the pressure p are provided, e.g. as data or on a display, wherein the thickness t′ can be determined by a not depicted method step, in which travel times of ultrasonic sound are determined for measuring the thickness t′ of the object 4.

Furthermore, method 40 comprises method step 47, in which the quality of the connection between the coupler 3 and the object 4 is checked. In particular, method step 47 may comprise analyzing spectra 31, 32, 33, 34 of ultrasonic sound reflected by interface 7.

Method 40 can be performed several times with different pressures p in order to determine the at least one property of the object of study.

FIG. 7 shows a method 50 for calibrating the palpator 1 schematically as a flow chart. Same reference signs are being used for elements, which correspond in function and/or structure to the elements of the previous exemplary embodiments. For the sake of brevity, only the differences from the exemplary embodiments of FIGS. 1 to 6 are discussed.

Method 50 starts with method step 51 in which palpator 1 may be put into operation.

In the following method step 52, coupler 3 is pressed against a calibration body, which may have comparable mechanical properties as coupler 3 and in particular have the same stiffness or elasticity. Alternatively, the calibration body may not be deformable by the pressure p.

In the following method step 53, ultrasonic sound is conducted through the coupler 3 and travel times of the ultrasonic sound are measured.

In method step 54, the pressure p is varied to known values. In method step 55, the known pressure values and the measured travel times are correlated with each other in order to calibrate the palpator. A calibration table may be stored in the evaluation apparatus 21.

1. An ultrasonic palpator (1) with an ultrasonic transducer (2) and an ultrasonic coupler (3) for coupling ultrasonic sound from the ultrasonic transducer (2) to an object of study (4), characterized in that the ultrasonic coupler (3) is elastically deformable.

2. The palpator (1) according to claim 1, characterized in that the coupler (3) has a Young's module between 1 and 10 N/mm² at between 20° C. and 36° C.

3. The palpator (1) according to claim 1, characterized in that the coupler (3) has an elasticity that essentially corresponds to the elasticity of cartilage.

4. The palpator (1) according to claim 1, characterized in that the coupler (3) is made of an elastomer.

5. The palpator (1) according to claim 1, characterized in that the coupler (3) is formed with an ultrasonic emitting section (16) that comprises a free emitting end (17) wherein the emitting section (16) has a cylindrical shape.

6. The palpator (1) according to claim 5, characterized in that the coupler (3) is formed with an ultrasonic receiving section (18) that is arranged between the emitting section (16) and the transducer (2), wherein the receiving section (18) is less deformable than the emitting section.

7. The palpator (1) according to claim 1, characterized in that the coupler (3) is repeatedly mountable to the palpator (1).

8. The palpator (1) according to claim 1, characterized by an ultrasonic sensor (2) that is connected to the coupler (3) in an ultrasonic conducting manner, wherein the palpator (1) comprises a spectrum analyzer (23) for analyzing the frequency spectrum of ultrasonic sound received by the ultrasonic sensor (2).

9. A measurement system (20) comprising a palpator (1) and an evaluation apparatus (21) that is connected to the palpator (1) in a measurement signal transmitting manner, characterized in that the palpator (1) is a palpator (1) according to claim 1.

10. The measurement system (20) according to claim 9, characterized in that the evaluation apparatus (21) is adapted to determine a pressure (p) that acts onto the coupler (3) by measuring travel times of ultrasonic sound through the coupler (3).

11. A kit for examining an object of study (4), with an ultrasonic palpator (1) having an ultrasonic transducer (2), characterized in that the kit comprises at least two ultrasonic couplers (3) for coupling ultrasonic sound from the transducer (2) into the object (4), wherein the couplers (3) are elastically deformable, have different elasticities and are exchangeably mountable to the palpator (1).

12. A method (40) for determining at least one property of an object of study (4), wherein ultrasonic sound is coupled into the object (4) via a coupling path (X, 43) and travel times of the ultrasonic sound through the object (4) are measured for determining the property, characterized in that a pressure (p) acting on the object (4) is measured by determining travel times of ultrasonic sound through the coupling path (X, 44), whose length (I) is changed by the pressure (p).

13. A method (40) for operating an ultrasonic palpator (1) for examining mechanical properties of an object of study (4), wherein ultrasonic sound is conducted through an ultrasonic coupler (3, 43) and a pressure (p) is exerted onto the object of study (4) by the coupler (3, 42), characterized in that the palpator (1) is a palpator according to claim 1 and in that the coupler (3) is elastically deformed by the pressure (p) and travel times of the ultrasonic sound required for travelling through the coupler (3) are used for determining the pressure (p).

14. The method (40) according to claim 13, characterized in that frequencies of ultrasonic sound received from the coupler (3) are used for determining the quality of a contact of the palpator (1) to the object of study (4).

15. A method (50) for calibrating a palpator (1), wherein the ultrasonic coupler (3) is pressed against a calibration body with a predetermined pressure (p, 52) and ultrasonic sound is conducted through the coupler (3) into the calibration body (53), characterized in that the pressure (p) is varied (54), wherein the coupler (3) is elastically deformed by the pressure (p) and travel times of ultrasonic sound required for travelling through the coupler (3) are correlated with the pressure (p, 55).
